(19) United States
(12) Patent Application Publication
(10) Pub. No.: US 2021/0123099 A1
(43) Pub. Date: Apr. 29, 2021

(54) HIGH MOLECULAR WEIGHT NUCLEIC ACID PREPARATION AND ANALYSIS METHODS

(71) Applicant: The Regents of the University of California, Oakland, CA (US)

(72) Inventor: Christopher Vollmers, Santa Cruz, CA (US)

(21) Appl. No.: 17/078,907

(22) Filed: Oct. 23, 2020

Related U.S. Application Data
(60) Provisional application No. 62/926,208, filed on Oct. 25, 2019.

(51) Int. Cl. C12Q 1/6872; C08F 220/56; C08B 37/00; G02B 6/10

(52) U.S. Cl. CPC C12Q 1/6872; G02B 6/107; C08B 37/0039; C08F 220/56

(57) ABSTRACT

Provided are methods of preparing high molecular weight nucleic acids for analysis. In certain embodiments, the methods comprise migrating nucleic acids comprising high molecular weight nucleic acids through a polymeric matrix, excising a portion of the polymeric matrix comprising high molecular weight nucleic acids, and isolating the high molecular weight nucleic acids from the excised polymeric matrix. The isolating comprises immobilizing the high molecular weight nucleic acids on particulate solid supports, and eluting the high molecular weight nucleic acids from the particulate solid supports. The methods may further comprise analyzing the isolated high molecular weight nucleic acids. According to some embodiments, the analyzing comprises sequence analysis of the isolated high molecular weight nucleic acids, e.g., using a nanopore- or zero mode waveguide (ZMW)-based sequencing device.

### CROSS-REFERENCE TO RELATED APPLICATIONS

This application claims the benefit of U.S. Provisional Patent Application No. 62/926,208, filed Oct. 25, 2019, which application is incorporated herein by reference in its entirety.

### INTRODUCTION

Recent advances in DNA sequencing have revolutionized the field of genomics, making it possible for even single research groups to generate large amounts of sequence data very rapidly and at a substantially lower cost. These high-throughput sequencing technologies make deep transcriptome sequencing and transcript quantification, whole genome sequencing and resequencing available to many more researchers and projects.

A variety of commercial high-throughput sequencing platforms exist and are described, e.g., in Metzker, M. L. (2010) Nat. Rev. Genet. 11:31-46, Morey et al. (2013) Mol. Genet. Metab. 110: 3-24, Reuter et al. (2015) Molecular Cell 58(4): 586-597, and elsewhere. In the Illumina platform, the sequencing process involves clonal amplification of adaptor-ligated DNA fragments on the surface of a glass slide. Bases are read using a cyclic reversible termination strategy, which sequences the template strand one nucleotide at a time through progressive rounds of base incorporation, washing, imaging, and cleavage. In this strategy, fluorescently labeled 3′-O-azidomethyl-dNTPs are used to pause the polymerization reaction, enabling removal of unincorporated bases and fluorescent imaging to determine the added nucleotide. Following scanning of the flow cell with a coupled-charge device (CCD) camera, the fluorescent moiety and the 3′ block are removed, and the process is repeated.

An emerging single-molecule strategy that has made significant progress in recent years is nanopore-based sequencing. Nanopore sequencing principally relies on the transition of DNA, RNA, or individual nucleotides through a small channel. A sequencing flow cell may include independent micro-wells, each containing a synthetic bilayer perforated by nanopores. Sequencing is accomplished by measuring characteristic changes in current that are induced as the bases are threaded through the pore by a molecular motor protein. Library preparation is minimal, involving fragmentation of DNA and ligation of adapters, and can be done with or without PCR amplification. The library design allows sequencing of both strands of DNA from a single molecule, which increases accuracy.

A difficulty in long sequencing reads of genomic DNA is delivery of only un-sheared long (>100 kb) DNA to the DNA sequencer. The presence of shorter DNA strands reduces throughput for the desired long strand reads. One of the challenges in this process is removing unwanted shorter DNA strands in the sequencing library. Size selection may be accomplished with expensive and time-consuming methodologies (e.g., the BluePippin™ DNA Size Selection System available from Sage Science), which also require relatively large microgram quantities of DNA. Simpler, rapid and less expensive methods for size selection are needed.

### SUMMARY

Provided are methods of preparing high molecular weight nucleic acids for analysis. In certain embodiments, the methods comprise migrating nucleic acids comprising high molecular weight nucleic acids through a polymeric matrix, excising a portion of the polymeric matrix comprising high molecular weight nucleic acids, and isolating the high molecular weight nucleic acids from the excised polymeric matrix. The isolating comprises immobilizing the high molecular weight nucleic acids on particulate solid supports, and eluting the high molecular weight nucleic acids from the particulate solid supports. The methods may further comprise analyzing the isolated high molecular weight nucleic acids. According to some embodiments, the analyzing comprises sequence analysis of the isolated high molecular weight nucleic acids, e.g., using a nanopore- or zero mode waveguide (ZMW)-based sequencing device.

### BRIEF DESCRIPTION OF THE FIGURES

FIG. 1 Plots showing the size distribution of sequence read lengths obtained from gel-extracted DNA prepared according to one embodiment of the present disclosure (right) and corresponding non-gel-extracted DNA (left).

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods of preparing high molecular weight nucleic acids for analysis. In certain embodiments, the methods comprise migrating nucleic acids comprising high molecular weight nucleic acids through a polymeric matrix, excising a portion of the polymeric matrix comprising high molecular weight nucleic acids, and isolating the high molecular weight nucleic acids from the excised polymeric matrix. The isolating comprises immobilizing the high molecular weight nucleic acids on particulate solid supports, and eluting the high molecular weight nucleic acids from the particulate solid supports.

Commercial long-read sequencing technologies (e.g., nanopore- and zero mode waveguide (ZMW)-based sequencing technologies provided by Oxford Nanopore Technologies Limited and Pacific Biosciences of California, Inc., respectively) require long DNA as input. There is currently no simple way to separate long and short DNAs. Existing methods are unreliable, low yield, and/or highly labor intensive. The methods of the present disclosure are based in part on the surprising discovery that excision of long DNA from a polymeric matrix paired with reversible particulate solid support immobilization-based isolation makes it possible to obtain highly pure high molecular weight DNA with minimal degradation thereof. Details regarding embodiments of the present disclosure will now be described.

By “high molecular weight nucleic acids” is meant nucleic acids that are 5 kilobases (kb) in length or greater. In certain embodiments, the high molecular weight nuclei acids are 6 kb in length or greater, 6.5 kb in length or greater, 7 kb in length or greater, 7.5 kb in length or greater, 8 kb in length or greater, 8.5 kb in length or greater, 9 kb in length or greater, 9.5 kb in length or greater, 10 kb in length or greater, 10.5 kb in length or greater, 11 kb in length or greater, 11.5 kb in length or greater, 12 kb in length or greater, 12.5 kb in length or greater, 13 kb in length or greater, 13.5 kb in length or greater, 14 kb in length or greater, 14.5 kb in length or greater, or 15 kb in length or greater.

The term “nucleic acid” refers to deoxyribonucleic acid (DNA), ribonucleic acid (RNA), single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid bases or to the nucleic acids as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

According to some embodiments, the high molecular weight nucleic acids are high molecular weight deoxyribonucleic acids (DNAs). High molecular weight DNAs of interest include, but are not limited to, high molecular weight genomic DNA (including high molecular weight genomic DNA fragments), high molecular weight mitochondrial DNA (mtDNA), high molecular weight complementary DNA (or “cDNA”, synthesized from any high molecular weight RNA or DNA of interest), high molecular weight recombinant DNA (e.g., high molecular weight plasmid DNA), or the like. In certain embodiments, the target nucleic acids are high molecular weight ribonucleic acids (RNAs), e.g., high molecular weight messenger RNAs (mRNAs).

The high molecular weight nucleic acids may be present in any nucleic acid sample of interest. In certain embodiments, the high molecular weight nucleic acids are present in a nucleic acid sample isolated from a single cell, a plurality of cells (e.g., cultured cells), a tissue, an organ, or an organism (e.g., bacteria, yeast, or the like). According to some embodiments, the nucleic acid sample is isolated from a cell(s), tissue, organ, and/or the like of an animal. In some embodiments, the animal is a mammal, e.g., a mammal from the genus Homo (e.g., a human), a rodent (e.g., a mouse or rat), a dog, a cat, a horse, a cow, or any other mammal of interest. In certain embodiments, the nucleic acid sample is isolated/obtained from a source other than a mammal, such as bacteria, yeast, insects (e.g., drosophila), amphibians (e.g., frogs (e.g., Xenopus)), viruses, plants, or any other non-mammalian nucleic acid sample source.

The nucleic acid sample may be from an extant organism or animal. In other embodiments, however, the nucleic acid sample may be from an extinct (or “ancient”) organism or animal, e.g., an extinct mammal, such as an extinct mammal from the genus Homo. According to some embodiments, the nucleic acid sample is obtained as part of a forensics analysis (e.g., a nucleic acid sample obtained from a crime scene, a victim of a crime, a crime suspect, and/or the like). In certain embodiments, the nucleic acid sample is obtained as part of a diagnostic analysis, e.g., from biopsy fluid or tissue (e.g., tumor biopsy tissue).

In certain embodiments, the nucleic acid sample comprises degraded DNA. Degraded DNA may be referred to as low-quality DNA or highly degraded DNA. Degraded DNA may be highly fragmented, and may include damage such as base analogs and abasic sites subject to miscoding lesions. For example, sequencing errors resulting from deamination of cytosine residues may be present in certain sequences obtained from degraded DNA, e.g., miscoding of C to T and G to A.

According to some embodiments, the nucleic acid sample is a cell-free nucleic acid sample, e.g., cell-free DNA, cell-free RNA, or both. Such cell-free nucleic acids may be obtained from any suitable source. In certain embodiments, the cell-free nucleic acids are from a body fluid sample selected from the group consisting of: whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In certain embodiments, the cell-free nucleic acids are cell-free fetal DNAs. According to some embodiments, the cell-free nucleic acids are circulating tumor DNAs. In certain embodiments, the cell-free nucleic acids comprise infectious agent DNAs. According to some embodiments, the cell-free nucleic acids comprise DNAs from a transplant.

The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject).

Cell-free nucleic acid often includes no detectable cells and may contain cellular elements or cellular remnants. Non-limiting examples of acellular sources for cell-free nucleic acid are described above. Obtaining cell-free nucleic acid may include obtaining a sample directly (e.g., collecting a sample, e.g., a test sample) or obtaining a sample from another who has collected a sample. Without being limited by theory, cell-free nucleic acid may be a product of cell apoptosis and cell breakdown, which provides basis for cell-free nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded.

Cell-free nucleic acid can include different nucleic acid species, and therefore is referred to herein as “heterogeneous” in certain embodiments. For example, a sample from a subject having cancer can include nucleic acid from cancer cells (e.g., tumor, neoplasia) and nucleic acid from non-cancer cells. In another example, a sample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a sample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid). In another example, heterogeneous cell-free nucleic acid may include nucleic acid from two or more subjects (e.g., a sample from a crime scene).

The nucleic acid sample may be a tumor nucleic acid sample (that is, a nucleic acid sample isolated from a tumor). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

Approaches, reagents and kits for isolating, purifying and/or concentrating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md.); the DNAzol®, ChargeSwitch®, Purelink®, GeneCatcher® nucleic acid isolation/purification kits by Life Technologies, Inc. (Carlsbad, Calif.); the NucleoMag®, NucleoSpin®, and NucleoBond® nucleic acid isolation/purification kits by Clontech Laboratories, Inc. (Mountain View, Calif.). In certain embodiments, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif.), and the NucleoSpin® FFPE kits by Clontech Laboratories, Inc. (Mountain View, Calif.).

When an organism, plant, animal, etc. from which the nucleic acid sample is obtained is extinct (or “ancient”), suitable strategies for recovering such nucleic acids are known and include, e.g., those described in Green et al. (2010) Science 328(5979): 710-722; Poinar et al. (2006) Science 311(5759): 392-394; Stiller et al. (2006) Proc. Natl. Acad. Sci. 103(37): 13578-13584; Miller et al. (2008) Nature 456(7220): 387-90; Rasmussen et al. (2010) Nature 463(7282): 757-762; and elsewhere.

According to some embodiments, prior to migrating the nucleic acids comprising high molecular weight nucleic acids through the polymeric matrix, the nucleic acid sample is prepared as described in International Patent Application No. PCT/US2019/028312 and Volden et al. (2018) PNAS 115(39): 9726-9731. Briefly, nucleic acids of a nucleic acid sample are circularized (e.g., using Gibson assembly), followed by amplification of the circularized nucleic acids by rolling circle amplification (e.g., using Phi29 polymerase and specific and/or random primers) to produce concatemers of the nucleic acids. The resulting concatemers comprising high molecular weight concatemers may be migrated through the polymeric matrix in accordance with the methods of the present disclosure.

Various approaches for migrating nucleic acids through a polymeric matrix may be employed when practicing the methods of the present disclosure. In certain embodiments, an approach that utilizes the negatively-charged nature of nucleic acids is employed. A non-limiting example of such an approach is electrophoresis, which refers to the migration and separation of charged molecules through a matrix under the influence of an electric field. The electric field is applied such that one end of the matrix has a positive charge and the other end has a negative charge. Because DNA and RNA are negatively-charged molecules, they migrate toward the positively charged end of the matrix. The molecules travel through the pores of the matrix at a speed that is inversely related to their lengths. Typically, the nucleic acid sample or aliquot thereof is placed in (e.g., pipetted into) a “well” of the polymeric matrix, where the well determines the position of a “lane” in which the nucleic acids placed in the well migrate toward the positively charged end of the matrix. One or more lanes of the matrix may be reserved for a standard mixture having a set of nucleic acids (“nucleic acid standards”) of known molecular weight. The standard mixture is separated at the same time as the nucleic acid sample, so that the standard mixture provides an indication of how far nucleic acids of a given molecular weight have traveled through the matrix. The matrix may also include a buffering agent including, but not limited to, Tris-acetate with ethylenediaminetetraacetic acid (EDTA) or Tris-borate with EDTA. According to some embodiments, the nucleic acids are migrated through the polymeric matrix by one-dimensional electrophoresis—that is, the nucleic acids are migrated in a single direction by virtue of the voltage being run in a single direction during the migrating. In certain embodiments, the nucleic acids are migrated through the polymeric matrix by multi-dimensional electrophoresis—that is, the nucleic acids are migrated in two or more directions by virtue of the voltage being periodically switched among two or more (e.g., three) directions during the migrating. For example, the voltage may be periodically switched among three directions: one that runs through the central axis of the matrix and two that run at an angle of 60 degrees either side. The pulse times may be equal or substantially equal for each direction resulting in a net forward migration of the nucleic acids. For extremely large nucleic acids (up to around 2 Mb), switching-interval ramps can be used that increases the pulse time for each direction over the course of a number of hours—e.g., increasing the pulse linearly from 10 seconds at 0 hours to 60 seconds at 18 hours. According to some embodiments, the nucleic acids are migrated through the polymeric matrix by pulsed-field electrophoresis. See, e.g., Sharma-Kuinkel et al. (2016) Methods Mol Biol. 1373: 117-130. Further details regarding electrophoresis approaches that may be employed when practicing the methods of the present disclosure are found, e.g., in DNA Electrophoresis (2013) Makovets, Svetlana (Ed.) ISBN 978-1-62703-565-1; and elsewhere. Apparatuses and reagents for electrophoresing nucleic acids are commercially available from, e.g., Bio-Rad Laboratories Inc., among others.

Any suitable polymeric matrix may be employed. In certain embodiments, the polymeric matrix comprises a gel-forming polymer. Non-limiting examples of gel-forming polymers that may be employed include those comprising agarose, polyacrylamide, starch, and any combination thereof. In certain embodiments, the polymeric matrix is an agarose gel. Agarose is a purified linear galactan hydrocolloid isolated from agar or agar-bearing marine algae. Structurally, it is a linear polymer consisting of alternating D-galactose and 3,6-anhydro-L-galactose units. According to some embodiments, the agarose is a low melting (LM) agarose, e.g., an agarose having a melting temperature of about ≤70° C., e.g., about ≤65.5° C. When the polymeric matrix is an agarose gel, the agarose may be present in any suitable percentage. In certain embodiments, the agarose is present in the gel at from 0.8% to 1.2%, e.g., from 0.9% to 1.1%, e.g., about 1%.

In other embodiments, the polymeric matrix comprises a non-gel-forming polymer. Non-limiting examples of non-gel-forming polymers that may be employed include those comprising linear polyacrylamide, poly(N, N-dimethylacrylamide), poly(hydroxyethylcellulose), poly(ethyleneoxide), poly(vinlyalcohol), and any combination thereof.

As summarized above, the methods of preparing high molecular weight nucleic acids for analysis comprise excising a portion of the polymeric matrix comprising high molecular weight nucleic acids. In certain embodiments, the excising comprises cutting (e.g., using a blade or the like) a portion of the polymeric matrix comprising high molecular weight nucleic acids to separate the portion from the remaining portion of the polymeric matrix. The portion to be excised may be identified by visualizing the high molecular weight nucleic acids in the polymeric matrix. A nucleic acid stain may be present in the polymeric matrix and/or present in the nucleic acid sample loaded into the well to facilitate visualization of the high molecular weight nucleic acids in the polymeric matrix. Non-limiting examples of suitable nucleic acid stains include SYBR® Gold nucleic acid gel stain (Thermo Fisher Scientific), SYBR® Green I nucleic acid gel stain (Sigma-Aldrich), and the like. A nucleic acid ladder (e.g., a DNA ladder or RNA ladder) may be run on the polymeric matrix for comparison to locate the portion of the polymeric matrix comprising the high molecular weight nucleic acids.

In some embodiments, multiple aliquots of the nucleic acid sample are run in separate lanes of the polymeric matrix, and the excising comprises excising high molecular weight nucleic acids from two or more of the lanes. The excised portions of the polymeric matrix may be pooled prior to the isolating step, or the high molecular weight nucleic acids from the excised portions of the polymeric matrix may be separately isolated.

As summarized above, isolating the high molecular weight nucleic acids from the excised polymeric matrix comprises immobilizing the high molecular weight nucleic acids on particulate solid supports. The term “solid support” means an insoluble material having a surface to which reagents or materials can be attached so that they can be readily separated from a solution. By “particulate solid supports” is meant a collection of solid supports having an average greatest dimension of 1000 micrometers (μm) or less. In some embodiments, the collection of solid supports has an average greatest dimension of 750 μm or less, 500 μm or less, 250 μm or less, 100 μm or less, 1 μm or less, 0.75 μm or less, 0.50 μm or less, 0.25 μm or less, or 0.1 μm or less. In certain embodiments, the particulate solid supports have an average greatest dimension of from about 0.50 μm to about 500 μm, e.g., from about 0.75 μm to about 250 μm, e.g., about 1 μm.

A variety of materials can be used as the particulate solid supports. Support materials include any material that can act as a support for attachment of the high molecular weight nucleic acids. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, metals, metalloids, magnetic materials, and any combinations thereof.

The particulate solid supports may be any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape.

In certain embodiments, the particulate solid supports are beads. As used herein, the term “bead” refers to a small mass that is generally spherical or spheroid in shape. According to some embodiments, a bead as used herein has an average diameter of from about 0.50 μm to about 500 μm, e.g., from about 0.75 μm to about 250 μm, e.g., about 1 μm.

Additionally, and for purposes herein, the particulate solid supports may be magnetically responsive, e.g., by virtue of comprising one or more paramagnetic and/or superparamagnetic substances, such as for example, magnetite. Such paramagnetic and/or superparamagnetic substances may be embedded within the matrix of the particulate solid supports, and/or may be disposed on an external and/or internal surface of the bead.

In certain embodiments, the particulate solid supports are particulate magnetic solid supports coated with a substance on their external surface that binds nucleic acids (e.g., DNA) non-specifically and reversibly. According to some embodiments, the substance comprises carboxyl groups that non-specifically and reversibly bind nucleic acids. A non-limiting example of such a substance is succinic acid. In certain embodiments, the particulate solid supports are solid phase reversible immobilization (SPRI) beads. SPRI beads such as AMPure XP® beads (Beckman Coulter Life Sciences) and protocols for using SPRI beads are known and readily available.

According to some embodiments, the isolating comprises hydrolyzing the excised polymeric matrix to produce polymeric matrix hydrolysis products, immobilizing the high molecular weight nucleic acids on the particulate solid supports, separating the particulate solid supports from the polymeric matrix hydrolysis products, and eluting the high molecular weight nucleic acids from the particulate solid supports. In certain embodiments, the polymeric matrix comprises agarose, and the hydrolyzing comprises digesting the agarose with an agarase enzyme. Suitable agarase enzymes that may be employed include, but are not limited to, β-Agarase I (available with protocols from, e.g., New England Biolabs, Inc.). According to some embodiments, the particulate solid supports are magnetic (e.g., paramagnetic), and separating the particulate solid supports from the polymeric matrix hydrolysis products comprises exposing the particulate solid supports to a magnet.

In certain embodiments, the molecular weight range of the high molecular weight nucleic acids to be immobilized on the particulate solid supports is selected by manipulating the ratio of the volume of particulate solid support buffer (e.g., PEG+salt) to the volume of the excised polymeric matrix. Lower particulate solid support (e.g., bead) buffer to excised polymeric matrix volume ratios correlate with higher molecular weights retained on the particulate solid supports, enabling size selection of higher molecular weight nucleic acids.

A variety of suitable approaches may be employed to elute the high molecular weight nucleic acids from the particulate solid supports. Such approaches may vary depending upon the nature of the reversible association between the high molecular weight nucleic acids and the surface of the particulate solid supports. In some embodiments, eluting the high molecular weight nucleic acids from the particulate solid supports comprises suspending the particulate solid support-high molecular weight nucleic acid complexes in water to dissociate the high molecular weight nucleic acids from the particulate solid supports.

A non-limiting example of a method of preparing high molecular weight nucleic acids for analysis according to the present disclosure is provided in the Experimental section below.

As summarized above, the methods of the present disclosure result in the preparation of high molecular weight nucleic acids for analysis. In certain embodiments, the excising comprises excising a portion of the polymeric matrix comprising high molecular weight nucleic acids of 5 kilobases (kb) in length or greater. When the excised portion of the polymeric matrix comprises high molecular weight nucleic acids of 5 kb in length or greater, according to some embodiments, 50% or more, 60% or more, 70% or more, 80% or more, or 90% or more of the isolated high molecular weight nucleic acids are 5 kb in length or greater. In certain embodiments, the excising comprises excising a portion of the polymeric matrix comprising high molecular weight nucleic acids of 10 kb in length or greater. When the excised portion of the polymeric matrix comprises high molecular weight nucleic acids of 10 kb in length or greater, according to some embodiments, 50% or more, 60% or more, 70% or more, 80% or more, or 90% or more of the isolated high molecular weight nucleic acids are 10 kb in length or greater.

According to some embodiments, the methods of the present disclosure of preparing high molecular weight nucleic acids for analysis further comprise analyzing the isolated high molecular weight nucleic acids. The isolated high molecular weight nucleic acids may be analyzed by a wide variety of types of analyses, including but not limited to, Southern analysis (to detect particular high molecular weight DNAs (e.g., high molecular weight genomic DNA fragments) of interest), Northern analysis (to detect particular high molecular weight RNAs (e.g., high molecular weight mRNAs) of interest), PCR analysis, and/or the like. In certain embodiments, the analyzing comprises analyzing the length of the isolated high molecular weight nucleic acids.

According to some embodiments, when the methods of the present disclosure of preparing high molecular weight nucleic acids for analysis further comprise analyzing the isolated high molecular weight nucleic acids, the analyzing comprises sequence analysis of the isolated high molecular weight nucleic acids. As will be appreciated with the benefit of the present disclosure, due to their high molecular weight nature, the nucleic acids prepared according to the present methods are particularly amenable to sequence analysis using a single molecule long-read sequencing system. Such systems include, but are not limited to: nanopore-based sequencing systems (e.g., a SmidgION, MinION, GridION, or PromethION nanopore-based sequencing system available from Oxford Nanopore Technologies Limited); and zero mode waveguide (ZMW)-based sequencing systems (e.g., a Sequel II ZMW-based sequencing system available from Pacific Biosciences of California, Inc.). Detailed design considerations and protocols for preparing nucleic acids (e.g., any necessary adapter addition, etc.), conducting nucleic acid sequencing runs, and analyzing the resulting sequencing data are provided by the manufacturers of such systems.

In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase's mass and its associated electrical field, the ionic current levels through the nanopore reveal the sequence of nucleobases in the translocating strand. A patch clamp, a voltage clamp, or the like, may be employed.

Suitable conditions for measuring ionic currents through transmembrane pores (e.g., protein pores, solid state pores, etc.) are known in the art. Typically, a voltage is applied across the membrane and pore. The voltage used may be from +2 V to −2 V, e.g., from −400 mV to +400 mV. The voltage used may be in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage may be in the range of from 100 mV to 240 mV, e.g., from 120 mV to 220 mV.

The methods are typically carried out in the presence of a suitable charge carrier, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or l-ethyl-3-methyl imidazolium chloride. Generally, the salt is present in the aqueous solution in the chamber. Potassium chloride (KCI), sodium chloride (NaCl) or cesium chloride (CsCI) may be used, for example. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. The salt concentration may be from 150 mM to 1 M. The methods are preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

In some embodiments, the rate at which the concatemer is exposed to the nanopore is controlled using a processive enzyme. Non-limiting examples of processive enzymes that may be employed include polymerases (e.g., a phi29 or other suitable polymerase) and helicases, e.g., a Hel308 helicase, a RecD helicase, a Tral helicase, a Tral subgroup helicase, an XPD helicase, or the like. The concatemer may be bound by the processive enzyme (e.g., by binding of the processive enzyme to a recognition site present in a sequencing adapter located at an end of the concatemer), followed by the resulting complex being drawn to the nanopore, e.g., by a potential difference applied across the nanopore. In other aspects, the processive enzyme may be located at the nanopore (e.g., attached to or adjacent to the nanopore) such that the processive enzyme binds the concatemer upon arrival of the concatemer at the nanopore.

The nanopore may be present in a solid-state film, a biological membrane, or the like. In some embodiments, the nanopore is a solid-state nanopore. In other embodiments, the nanopore is a biological nanopore. The biological nanopore may be, e.g., an alpha-hemolysin-based nanopore, a Mycobacterium smegmatis porin A (MspA)-based nanopore, or the like.

Details for obtaining raw sequencing reads of nucleic acid molecules using nanopores are described, e.g., in Feng et al. (2015) Genomics, Proteomics & Bioinformatics 13(1): 4-16. Nanopore-based sequencing systems are available and include the SmidgION, MinION, GridION, and PromethION nanopore-based sequencing systems available from Oxford Nanopore Technologies Limited. Detailed design considerations and protocols for performing nucleic acid sequencing are provided with such systems.

In zero mode waveguide (ZMW)-based sequence analysis, the ZMW is a nanoscale-sized well that serves as an optical confinement that allows observation of individual polymerase molecules. As a result, nucleotide incorporation events provide observation of an incorporating nucleotide analog that is readily distinguishable from non-incorporated nucleotide analogs. For a description of ZMWs and their application in nucleic acid sequencing, see, e.g., U.S. Patent Application Publication No. 2003/0044781 and U.S. Pat. No. 6,917,726, each of which is incorporated herein by reference in its entirety for all purposes. See also Levene et al. (2003) “Zero-mode waveguides for single-molecule analysis at high concentrations” Science 299: 682-686, Eid et al. (2009) “Real-time DNA sequencing from single polymerase molecules” Science 323: 133-138, and U.S. Pat. Nos. 7,056,676, 7,056,661, 7,052,847, 7,033,764, and 7,907,800, the full disclosures of which are incorporated herein by reference in their entirety for all purposes.

Aspects of the present disclosure further include methods of analyzing high molecular weight nucleic acids. In certain embodiments, the methods include analyzing high molecular weight nucleic acids prepared according to any of the methods of preparing high molecular weight nucleic acids for analysis of the present disclosure. The high molecular weight nucleic acids may be analyzed by a wide variety of types of analyses, including but not limited to,

Southern analysis (to detect particular high molecular weight DNAs (e.g., high molecular weight genomic DNA fragments) of interest), Northern analysis (to detect particular high molecular weight RNAs (e.g., high molecular weight mRNAs) of interest), PCR analysis, and/or the like. In certain embodiments, the analyzing comprises analyzing the length of the isolated high molecular weight nucleic acids.

According to some embodiments, the analyzing comprises sequence analysis of the high molecular weight nucleic acids. For example, the high molecular weight nucleic acids may be analyzed using a single molecule long-read sequencing system, non-limiting examples of which include nanopore-based sequencing systems (e.g., a SmidgION, MinION, GridION, or PromethION nanopore-based sequencing system available from Oxford Nanopore Technologies Limited); and zero mode waveguide (ZMW)-based sequencing systems (e.g., a Sequel II ZMW-based sequencing system available from Pacific Biosciences of California, Inc.), as described hereinabove.

### Kits

The present disclosure also provides kits. In some embodiments, the kits of the present disclosure provide one or more reagents that find use in practicing the methods of preparing high molecular weight nucleic acids for analysis of the present disclosure. For example, such kits may include a reagent (e.g., agarose, such as low melt agarose) useful for forming a polymeric matrix; a reagent (e.g., an agarase enzyme, such as β-Agarase I) useful for hydrolyzing the excised polymeric matrix comprising the high molecular weight nucleic acids; a particulate solid support (e.g., magnetic beads capable of reversibly immobilizing high molecular weight nucleic acids, such as SPRI beads); one or more other reagents and/or buffers that find use in one or more of the migrating, excising and/or isolating steps of the present methods, and any combination thereof.

Components of the kits may be present in separate containers, or multiple components may be present in a single container. A suitable container includes a single tube (e.g., vial), one or more wells of a plate (e.g., a 96-well plate, a 384-well plate, etc.), or the like.

Any of the kits of the present disclosure may include instructions for using the components therein, e.g., instructions for performing any of the methods of preparing high molecular weight nucleic acids for analysis of the present disclosure. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:

1. A method of preparing high molecular weight nucleic acids for analysis, comprising: migrating nucleic acids comprising high molecular weight nucleic acids through a polymeric matrix;

excising a portion of the polymeric matrix comprising high molecular weight nucleic acids; and

isolating the high molecular weight nucleic acids from the excised polymeric matrix, wherein the isolating comprises:
immobilizing the high molecular weight nucleic acids on particulate solid supports; and
eluting the high molecular weight nucleic acids from the particulate solid supports.
   2. The method of embodiment 1, wherein the high molecular weight nucleic acids are high molecular weight deoxyribonucleic acids (DNA).
   3. The method of embodiment 1, wherein the high molecular weight nucleic acids are high molecular weight ribonucleic acids (RNA).
   4. The method of any one of embodiments 1 to 3, wherein the migrating comprises electrophoresing the nucleic acids through the polymeric matrix.
   5. The method of any one of embodiments 1 to 4, wherein the polymeric matrix comprises a gel-forming polymer.
   6. The method of embodiment 5, wherein the gel-forming polymer comprises agarose, polyacrylamide, starch, and any combination thereof.
   7. The method of any one of embodiments 1 to 4, wherein the polymeric matrix comprises a non-gel-forming polymer.
   8. The method of embodiment 7, wherein the non-gel-forming polymer comprises linear polyacrylamide, poly(N, N-dimethylacrylamide), poly(hydroxyethylcellulose), poly(ethyleneoxide), poly(vinlyalcohol), and any combination thereof.
   9. The method of any one of embodiments 1 to 8, wherein the isolating comprises:

hydrolyzing the excised polymeric matrix to produce polymeric matrix hydrolysis products;

immobilizing the high molecular weight nucleic acids on the particulate solid supports;

separating the particulate solid supports from the polymeric matrix hydrolysis products; and

eluting the high molecular weight nucleic acids from the particulate solid supports.

10. The method of embodiment 9, wherein the polymeric matrix comprises agarose, and wherein the hydrolyzing comprises digesting the agarose with an agarase enzyme.
11. The method of embodiment 9 or embodiment 10, wherein the particulate solid supports are magnetic, and wherein separating the particulate solid supports from the polymeric matrix hydrolysis products comprises exposing the particulate solid supports to a magnet.
12. The method of any one of embodiments 1 to 11, wherein the excising comprises excising a portion of the polymeric matrix comprising high molecular weight nucleic acids of 5 kilobases in length or greater.
13. The method of embodiment 12, wherein 50% or more, 60% or more, 70% or more, 80% or more, or 90% or more of the isolated high molecular weight nucleic acids are 5 kilobases in length or greater.
14. The method of any one of embodiments 1 to 11, wherein the excising comprises excising a portion of the polymeric matrix comprising high molecular weight nucleic acids of 10 kilobases in length or greater.
15. The method of embodiment 14, wherein 50% or more, 60% or more, 70% or more, 80% or more, or 90% or more of the isolated high molecular weight nucleic acids are 10 kilobases in length or greater.
16. The method of any one of embodiments 1 to 15, further comprising analyzing the isolated high molecular weight nucleic acids.
17. The method of embodiment 16, wherein the analyzing comprises analyzing the length of the isolated high molecular weight nucleic acids.
18. The method of embodiment 16 or embodiment 17, wherein the analyzing comprises sequence analysis of the isolated high molecular weight nucleic acids.
19. The method of any one of embodiments 16 to 18, wherein the analyzing comprises analyzing the isolated high molecular weight nucleic acids using a nanopore device.
20. The method of any one of embodiments 16 to 18, wherein the analyzing comprises analyzing the isolated high molecular weight nucleic acids using a zero mode waveguide (ZMW) device.
21. A method of analyzing high molecular weight nucleic acids, comprising:

analyzing high molecular weight nucleic acids prepared according to the method of any one of embodiments 1 to 15.

22. The method of embodiment 21, wherein the analyzing comprises analyzing the length of the high molecular weight nucleic acids.
23. The method of embodiment 21 or embodiment 22, wherein the analyzing comprises sequence analysis of the high molecular weight nucleic acids.
24. The method of any one of embodiments 21 to 23, wherein the analyzing comprises analyzing the high molecular weight nucleic acids using a nanopore device.
25. The method of any one of embodiments 21 to 23, wherein the analyzing comprises analyzing the isolated high molecular weight nucleic acids using a zero mode waveguide (ZMW) device.

The following examples are offered by way of illustration and not by way of limitation.

### Experimental

### Example 1—Combination Gel-Extraction and Bead-Based Isolation Methods of Preparing Nucleic Acids for Analysis

In this example, gel extraction and bead-based nucleic acid isolation were combined to prepare high molecular weight DNA for analysis. The combination of these two approaches unexpectedly resulted in highly pure, high molecular weight, minimally degraded DNA.

In this example, DNAs of a DNA sample were circularized using Gibson assembly, followed by amplification of the circularized nucleic acids by rolling circle amplification using Phi29 polymerase to produce concatemers of the DNAs, as described in Volden et al. (2018) PNAS 115(39): 9726-9731. One aliquot of the resulting concatemers was directly sequenced on a MinION sequencing system. A second aliquot was subjected to the following combination gel-extraction and bead-based isolation protocol:

### Migration by Gel Electrophoresis

1. Run DNA on 1% low melt agarose gel using Sybr-Gold® dye for visualizing the DNA. Load as many wells as needed.

2. Excise main band visible on gel (if not visible under normal room lighting, use blue light box).

3. Combine 4-5 slices into a 2 ml tube. Weigh tube on scale tared with empty 2 ml tube.

4. Assume that weight-volume, e.g., if 300 μg assume 300 μl volume.

### Agarose Digestion

This procedure will digest up to 300 μl of 1% low melting point agarose. For larger volumes, adjust enzyme accordingly.

1. Incubate 300 μl of gel slice with 2 volumes of 1× β-Agerase I Buffer on ice for 20 minutes.

2. Remove the remaining buffer and melt the agarose by incubation at 65° C. for 10 minutes.

3. Cool to 42° C. for 10 mins.

4. Add 1 unit (2 μl) of β-Agarase I.

5. Incubate at 42° C. for 1 hour.

### DNA Isolation

1. Chill on ice for 15 minutes.

2. Centrifuge at 15,000× g for 10 minutes to pellet any remaining undigested carbohydrates.

3. Remove the DNA-containing supernatant.

4. Combine samples as is convenient. Final volume should be <1000 ul.

5. Add 1 volume of SPRI beads (could be large volume)

6. Incubate for 5 min

7. Put on magnet and wait for solution to clear

8. Wash twice with 70% EtOH

9. Remove all EtOH

10. Add 30 μl (adjust if appropriate) of H₂O

11. Incubate at 37° C. for 10 min

12. Put on magnet and retrieve high molecular weight DNAs.

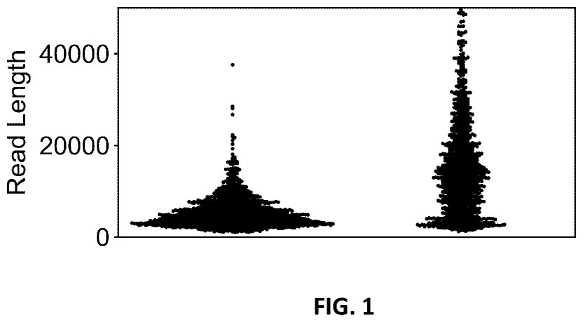

The high molecular weight DNAs prepared by combined gel-extraction and bead-based isolation were sequenced on a MinION sequencing system. Shown in FIG. 1 are plots showing the size distribution of sequence read lengths obtained from the gel-extracted and bead isolated DNA (right) and the corresponding directly sequenced non-gel-extracted DNA of the first aliquot (left). Read length units on the y-axis are in bases (e.g., “20000”=20 kb and “40000”=40 kb). As is apparent from the plots, the combination of gel extraction and bead isolation surprisingly resulted in highly pure, high molecular weight, minimally degraded DNA.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of preparing high molecular weight nucleic acids for analysis, comprising:
migrating nucleic acids comprising high molecular weight nucleic acids through a polymeric matrix;
excising a portion of the polymeric matrix comprising high molecular weight nucleic acids; and
isolating the high molecular weight nucleic acids from the excised polymeric matrix, wherein the isolating comprises:
immobilizing the high molecular weight nucleic acids on particulate solid supports; and
eluting the high molecular weight nucleic acids from the particulate solid supports.

2. The method of claim 1, wherein the high molecular weight nucleic acids are high molecular weight deoxyribonucleic acids (DNA).

3. The method of claim 1, wherein the migrating comprises electrophoresing the nucleic acids through the polymeric matrix.

4. The method of claim 1, wherein the polymeric matrix comprises a gel-forming polymer.

5. The method of claim 4, wherein the gel-forming polymer comprises agarose, polyacrylamide, starch, and any combination thereof.

6. The method of claim 1, wherein the polymeric matrix comprises a non-gel-forming polymer.

7. The method of claim 6, wherein the non-gel-forming polymer comprises linear polyacrylamide, poly(N, N-dimethylacrylamide), poly(hydroxyethylcellulose), poly(ethyleneoxide), poly(vinlyalcohol), and any combination thereof.

8. The method of claim 1, wherein the isolating comprises:
hydrolyzing the excised polymeric matrix to produce polymeric matrix hydrolysis products;
immobilizing the high molecular weight nucleic acids on the particulate solid supports;
separating the particulate solid supports from the polymeric matrix hydrolysis products; and
eluting the high molecular weight nucleic acids from the particulate solid supports.

9. The method of claim 8, wherein the polymeric matrix comprises agarose, and wherein the hydrolyzing comprises digesting the agarose with an agarase enzyme.

10. The method of claim 8, wherein the particulate solid supports are magnetic, and wherein separating the particulate solid supports from the polymeric matrix hydrolysis products comprises exposing the particulate solid supports to a magnet.

11. The method of claim 1 wherein the excising comprises excising a portion of the polymeric matrix comprising high molecular weight nucleic acids of 5 kilobases in length or greater.

12. The method of claim 11, wherein 50% or more, 60% or more, 70% or more, 80% or more, or 90% or more of the isolated high molecular weight nucleic acids are 5 kilobases in length or greater.

13. The method of claim 1, wherein the excising comprises excising a portion of the polymeric matrix comprising high molecular weight nucleic acids of 10 kilobases in length or greater.

14. The method of claim 13, wherein 50% or more, 60% or more, 70% or more, 80% or more, or 90% or more of the isolated high molecular weight nucleic acids are 10 kilobases in length or greater.

15. The method of claim 1, further comprising analyzing the isolated high molecular weight nucleic acids.

16. A method of analyzing high molecular weight nucleic acids, comprising analyzing high molecular weight nucleic acids prepared according to the method of claim 1.

17. The method of claim 16, wherein the analyzing comprises analyzing the length of the high molecular weight nucleic acids.

18. The method of claim 16, wherein the analyzing comprises sequence analysis of the high molecular weight nucleic acids.

19. The method of claim 16, wherein the analyzing comprises analyzing the high molecular weight nucleic acids using a nanopore device.

20. The method of claim 16, wherein the analyzing comprises analyzing the isolated high molecular weight nucleic acids using a zero mode waveguide (ZMW) device.
